(19) United States
(12) Patent Application Publication
(10) Pub. No.: US 2002/0128524 A1
(43) Pub. Date: Sep. 12, 2002

(54) Purification of dihydroxydiphenyl

(76) Inventors: Silke Kratschmer, Krefeld (DE); Hans Deml, Krefeld (DE)

Correspondence Address: BAYER CORPORATION, PATENT DEPARTMENT, 100 BAYER ROAD, PITTSBURGH, PA, 15205, US

(21) Appl. No.: 10/090,914

(22) Filed: Mar. 5, 2002

(30) Foreign Application Priority Data
Mar. 9, 2001 (DE) 10111289.0

(51) Int. Cl. C08G 64/00; C07C 39/02

(52) U.S. Cl. 568/743; 528/196

(57) ABSTRACT

A method of purifying dihydroxydiphenyl (1),

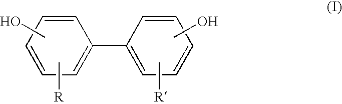

wherein

R and R′, independently of each other, denotes H or a C₁-C₄ alkyl radical, is disclosed. The pure dihydroxydiphenyl thus produced is suitable for preparing polycarbontes.

### FIELD OF THE INVENTION

The invention relates to a method for preparing pure dihydroxydiphenyl.

### SUMMERY OF THE INVENTION

This application relates to a method of purifying dihydroxydiphenyl (I),

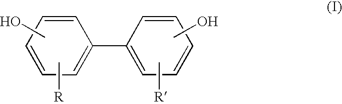

wherein

R and R′, independently of each other, denote H or a C₁-C₄ alkyl radical, preferably H.

### BACKGROUND OF THE INVENTION

Dihydroxydiphenyl, particularly 4,4′-dihydroxydiphenyl, is used for the production of various polymers. For use in copolycarbonates in particular, it is advantageous if the raw materials are as pure as possible so that no foreign substances contaminate the polymer and thus have a negative effect on the properties of the product, such as the color or the thermal stability thereof, for example. In addition, it is known that dark-colored raw materials also result in colored polycarbonates. Therefore, the intrinsic color of the raw materials used should be as light as possible.

The purification of 4,4′-dihydroxydiphenyl has already been described in EP A 349 382. In the method which is described there, the crude product which results from the production of dihydroxydiphenyl is esterified with an acetic acid derivative in the presence of a strong acid, and is crystallized out. An esterification operation means an additional reaction step, however, and is therefore to be avoided. For procedures comprising the direct recrystallization of dihydroxydiphenyl, this circumvention is avoided by the formation of a derivative. This procedure is described in JP A 3 099 031 and in JP A 4 005 251, for example. In addition to alcohols or ketones, however, toxic solvents such as phenol or dimethylformamide are used here, the effects of which likewise have to be avoided. In GB A 2 071 090, the alkali metal salt of dihydroxydiphenyl is purified by activated carbon and 4,4′-dihydroxydiphenyl is subsequently obtained by acidification. However, this procedure necessitates 7-40% by weight of activated carbon with respect to the diphenyl solution. Moreover, this method is only of use if the alkali metal salt is contained due to the method of synthesis employed and can be purified directly, but is of no use if dihydroxydiphenyl is obtained commercially, for example.

A common feature of all recrystallization methods is that they consume large amounts of aqueous and alcoholic solvents with respect to the dihydroxydiphenyl used, in order to achieve a purification effect. In addition to activated carbon, phenol or DMF, a considerable amount of solvent thus occurs as a waste product in these methods, and is accompanied by high costs of disposal.

The object of the present application is thus to identify a method in which the amounts of solvents arising remain as small as possible, and in which further reaction steps and solvents which are harmful to health are avoided.

### DETAILED DESCRIPTION OF THE INVENTION

The present application relates to a method of purifying dihydroxydiphenyl, characterized in that recrystallization is effected without further reaction steps and without toxic solvents, whilst the amounts of waste are small.

The purification of dihydroxydiphenyl, particularly 4,4′-dihydroxydiphenyl, is effected in short chain ketones as solvents, such as acetone, butanone or pentanone, preferably acetone. The purification operation can optionally be conducted using activated carbon, wherein the use of activated carbon is preferred.

Purification is preferably carried out at temperatures of 56 to 102° C., depending on the solvent. A temperature is preferred which is as low as possible but at which the selected solvent just boils.

1.5-4 liters of solvent are used for the purification of 350 kg dihydroxydiphenyl. 2 liters of solvent are most preferably used. This solvent can be repeatedly re-used after recrystallization, so that it is also possible, for example, to purify 5 kg dihydroxydiphenyl using only 6 liters of acetone, whilst 10-56 liters of acetone would be consumed without reuse of the solvent. If activated carbon is added, it is preferably used in an amount of 1.5-5 g, most preferably 3 g.

Dihydroxydiphenyls which are purified in this manner are distinguished by their considerably improved intrinsic color. This also has a positive effect on further reactions, e.g. for the production of copolycarbonates.

The method according to the invention may be carried out continuously or batch-wise, batch-wise operation is preferred.

According to one preferred embodiment, the solvent is placed in a vessel, optionally with activated carbon, and heated to boiling. The dihydroxydiphenyl is added with stirring, and the batch is stirred for 30-70 minutes, preferably for 30-45 minutes. The dihydroxydiphenyl should dissolve completely in the course of this procedure. The solution is subsequently filtered and brought to room temperature. The dihydroxydiphenyl which then precipitates out is filtered off under suction, washed once with solvent, and dried.

Dihydroxydiphenyls which are purified in this manner are particularly suitable for the production of polycarbonates, especially for the production of polycarbonates by the melt transesterification process.

As is known, the melt transesterification process employs starting materials comprising aromatic diphenols, diaryl esters of carbonic acid, and optionally branching agents and/or monophenols.

Further details of the melt transesterification process are described in the literature (see Hermann Schnell, Chemistry and Physics of Polycarbonates, Polymer Reviews, Volume 9, 1964, pages 44 to 51, DE-A-1 031 512, U.S. Pat. No. 3,022,272, U.S. Pat. No. 5,340,905 and U.S. Pat. No. 5,399,659).

Thermoplastic polycarbonates which are produced using dihydroxydiphenyls which may be obtained according to the invention possess a light intrinsic color. Moreover, copolycarbonates based on 4,4′-dihydroxydiphenyl and 2,2-bis(4-hydroxyphenyl) propane are known from JP 5 117 382, and are described in EP-A1 0 544 4 07, U.S. Pat. No. 5,470,938, U.S. Pat. No. 5,532,324 and U.S. Pat. No. 5,401,826 as being particularly resistant to chemicals, thermally resistant, and are flame resistant, whilst possessing the same mechanical properties and transparency as commercially available polycarbonates produced from pure bisphenol.

It is possible to use the polycarbonates which are produced in this manner, in the form of very different moldings in all industrial situations where thermoplastic polycarbonates have already been used hitherto, for instance in electrical engineering, as lamp covers, as safety panels or as optical data storage media such as CD material.

### EXAMPLES

The color number was determined as the difference between the extinction at 420 nm and at 700 nm in dichloromethane, at a concentration of 2.4 g/50 ml and at a layer thickness of 10 cm.

The color number of the crude 4,4′-dihydroxydiphenyl used was 0.062.

### Example 1

2 liters of acetone were placed, together with 3 g of activated carbon, in a 4-liter round bottomed flask fitted with a bottom outlet, a stirrer, a thermometer and a reflux condenser, and were heated to 58° C. 350 g 4,4′-dihydroxydiphenyl were added with stirring, and the batch was stirred for about 30-45 minutes until the dihydroxydiphenyl had dissolved. The solution was filtered via a doubly-folded filter. The dihydroxyphenyl crystallized out of the solution again at room temperature, was filtered off through a Buchner funnel and washed once with acetone, and was then dried in a vacuum drying oven for 3-4 hours at 80° C.

After filtering off the diphenyl under suction, the mother liquor could be used again for recrystallization.

The color number of the 4,4′-dihydroxydiphenyl was 0.010.

### Example 2

As in Example 1, except that no activated carbon was added.

The color number of the 4,4′-dihydroxydiphenyl was 0.022.

### Comparative Example 1

As in Example 1, except that acetic acid was used as the solvent.

The color number of the 4,4′-dihydroxydiphenyl was 0.036.

### Examples of Use

The relative solution viscosity was determined in dichloromethane at a concentration of 5 g/l at 25° C.

### Example of Use 1

A polycarbonate comprising 40 mol % 4,4′-dihydroxydiphenyl from Example 1 and 60 mol % bisphenol A was produced in a melt transesterification process. The product had a relative solution viscosity of 1.282. The color number of the polycarbonate was 0.050.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A method of purifying dihydroxydiphenyl comprising dissolving dihydroxydiphenyl in a short chain ketone solvent and recrystallizing the dihydroxydiphenyl and excluding additional reaction steps and toxic solvents.

2. The method according to claim 1 wherein ketones is a member selected from the group consisting of acetone, butanone and pentanone.

3. The method according to claim 1 wherein the ketone is acetone.

4. The method according to claim 1 wherein solvent further contains activated carbon.

5. The method according to claim 1 wherein the solvent is continuously recycled.

6. The method according to claim 1 wherein the dihydroxydiphenyl is 4,4′-dihydroxydiphenyl.

7. A method of using the dihydroxydiphenyls prepared by the method of claim 1 comprising producing polycarbonate.
